(19) United States
(12) Patent Application Publication
(10) Pub. No.: US 2002/0142306 A1
(43) Pub. Date: Oct. 3, 2002

(54) Method of transferring molecules to a film laminate

(75) Inventors: Patrick L. Coleman, Minneapolis, MN (US); Kurt J. Halverson, Lake Elmo, MN (US); James I. Hembre, Plymouth, MN (US); Sanjay L. Patil, Minneapolis, MN (US); Anila Prabhu, Woodbury, MN (US); Raj Rajagopal, Woodbury, MN (US); Jerald K. Rasmussen, Stillwater, MN (US); Barbara C. Swenson, North St. Paul, MN (US)

Correspondence Address: Attention: Christopher D. Gram, Office of Intellectual Property Counsel, 3M Innovative Properties Company, P.O. Box 33427, St. Paul, MN, 55133-3427, US

(73) Assignee: 3M Innovative Properties Company

(21) Appl. No.: 09/819,317

(22) Filed: Mar. 28, 2001

(51) Int. Cl. C12Q 1/68; B05D 3/00

(52) U.S. Cl. 435/6; 427/2.11

(57) ABSTRACT

A method for transferring molecules from a matrix to a laminate comprising a shrinkable polymeric film and a surface coating is disclosed.

### BACKGROUND

Analysis and detection of biological molecules typically involves placing a sample onto an immobilizing membrane and then performing steps to detect the presence of or quantitate one or more particular biological molecules in the sample. A sample may be spotted directly onto the immobilizing membrane or transferred from a matrix to the immobilizing membrane by blotting. Such a transfer may be necessary because the matrix can be unsuited for many of the biological or chemical assays known to those skilled in the art. The transfer may be passive or energy-driven, such as by an electric current. Once the sample has been transferred to the membrane, the desired assay can be performed on the immobilized sample.

Methods of transferring biological molecules to immobilizing membranes are known in the art. For example, polynucleotide sequences may be transferred from a gel made of agarose or polyacrylamide to a cellulose-derived or nylon membrane. Similarly, proteins may be transferred from an SDS-polyacrylamide gel to a cellulose-derived or nylon membrane. Immobilizing membranes made from nylon or cellulose-derived materials are porous and permit the transfer of polynucleotides or proteins through a variety of processes, some of which are energy independent and some of which, such as electroblotting, are energy-driven.

Many assays performed on biological molecules can be performed on a miniaturized scale. Many of these assays involve expensive and oftentimes difficult to obtain samples and reagents. Accordingly, assays performed on a miniaturized scale are desirable because they may dramatically reduce the amount of sample and reagents required for performing the assay. Miniaturized assays are especially desired when an expensive or limited sample can be concentrated, thereby reducing the amount of the sample required for the assay while simultaneously increasing the sensitivity, accuracy or efficiency of the assay. In addition to the reduction of volume, miniaturization allows hundreds or thousands of assays to be performed simultaneously.

A heat-shrinkable film such as that disclosed in International Publication No. WO 99/53319, published Oct. 21, 1999, permits samples to be concentrated for miniaturized assays. What is needed is a method of transferring molecules from a matrix to a shrinkable film.

### SUMMARY

The present invention provides a method for transferring a sample of biological molecules from a matrix to an immobilizing composite that is a laminate comprising a shrinkable film. Because the laminate is shrinkable, the transferred sample may be concentrated for use in a miniaturized assay.

The present invention provides a method of transferring molecules from a matrix to a laminate comprising: providing one or more molecules positioned within a matrix, contacting the matrix with a laminate comprising a shrinkable film and a hydrogel coating, and causing the one or more molecules to be transferred from the matrix to the laminate.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### DEFINITIONS

For purposes of this invention, the following definitions shall have the meanings set forth.

“A” or “an” refers to one or more of the recited elements.

“Affix” shall include any mode of attaching biological molecules to a substrate. Such modes shall include, without limitation, covalent and ionic bonding, adherence, such as with an adhesive, physical entrapment, and adsorption. This may or may not require the use of linking agents.

“Density” shall mean a measure of quantity per unit projected area of a substrate, such as, for example, molecules per square centimeter.

“Heat-relaxable” or “heat-shrinkable” shall mean, in the context of a material such as a substrate, that the material undergoes some relaxation or shrinkage in at least one dimension in response to the transmission of thermal energy into the material.

“Linking agent” shall mean any chemical species capable of affixing a “Molecule” to a substrate. Linking agents can be covalently bonded to the substrate or provided by a polymeric coating thereon.

“Molecule” shall be construed broadly to mean any molecule, compound, composition or complex, either naturally occurring or synthesized, to be detected or measured in or separated from a sample of interest. Molecules include, without limitation, polypeptides, fatty acids, polynucleotides, carbohydrates, polysaccharides, hormones, steroids, lipids, vitamins, bacteria, viruses, pharmaceuticals, and metabolites.

“Polynucleotide” shall mean any polymer of nucleotides without regard to its length. Thus, for example, ribonucleotides and deoxyribonucleotides are each included in the definition of polynucleotide as used herein, whether in single- or double-stranded form. A polynucleotide, as used herein, may be obtained directly from a natural source or may be synthesized using recombinant, enzymatic or chemical techniques. A polynucleotide may be linear or circular in topology and can be, for example, a vector such as an expression vector, cloning vector or any type of plasmid, or any fragment thereof.

“Polypeptide” shall mean any polymer of amino acids without regard to its length. Thus, for example, the terms peptide, oligopeptide, protein, enzyme, and fragments thereof are all included within the definition of polypeptide as used herein. The term also includes polypeptides that have been modified by post-expression or synthetic processes yielding, for example, glycosylated, acetylated, phosphorylated polypeptides, or peptide nucleic acids. Accordingly, a polypeptide may be obtained directly from a natural source or may be synthesized using enzymatic or chemical techniques.

“Polysaccharide” shall mean any polymer of saccharides without regard to its size. The term also includes classes of molecules that are polymers of saccharides in combination with other monomers such as amino acids, nucleotides, and any polymers thereof. Such classes of molecules include, but are not limited to, glycosaminoglycans, proteoglycans and glycolipids.

“Projected surface area” shall mean the surface area for a surface as is calculated with respect to the plane encompassing the “x” and “y” axes of the surface.

“Recoverable” means, in the context of a material, such as a substrate, that the material is stretched and capable of subsequently recovering at least one dimension, preferably to substantially its original size.

“Relaxable” shall mean, in the context of a material such as a substrate, that the material is capable of relaxing or shrinking, in at least one dimension. Preferably, shrinkage occurs by at least about 10%.

“Shrinkable,” “shrinking” or “shrunk” shall mean, in the context of a material such as a substrate, that the material is capable of being, is, or has been decreased in its length in at least one dimension, whether by recovery, relaxation, or any other means.

“Topographical surface area” shall mean the surface area of a surface as is calculated with respect to the planes encompassing the “x”, “y” and “z” axes of the surface, or in other words, a measurement of the surface features of the coating.

“Undulations” or “undulated” shall mean convoluted, wave-like forms. For purposes of this invention, it is preferred that an undulated surface includes undulations that do not form a regular pattern. “Undulations” or “undulated” does not include structures such as reservoirs or microwells that are created by methods such as for example printing, embossing, casting, molding, laserscribing, photolithography, etching, mechanical scratching, or scoring.

### BRIEF DESCRIPTION OF THE DRAWINGS

FIG. 1a is a side view of the laminate used in the method of present invention prior to relaxation of the substrate thereof.

FIG. 1b is a side view of the laminate of FIG. 1a subsequent to relaxation of the substrate thereof.

### DETAILED DESCRIPTION OF THE INVENTION

The present invention provides a method for transferring molecules from a matrix to an immobilizing composite that is a laminate comprising a shrinkable film. Generally, the method includes providing a sample of molecules in a matrix such as a matrix useful for separating molecules. The method also includes providing an immobilizing composite that is a laminate comprising a shrinkable film such as a polyethylene shrink film. The molecules provided in the matrix are transferred to the laminate by a process such as passive blotting or electrophoretic transfer, namely electroblotting, although these are not necessarily the only possible transfer processes. The transfer of molecules from a matrix to a laminate in this manner is surprising because these transfer techniques have not previously been demonstrated using a laminate such as that used in the claimed method as an immobilizing composite.

The Matrix

The matrix of the claimed method can be any matrix suitable for separating molecules. Such separation can be based on differences in the size, shape, electrical charge or any other physical or chemical property of the molecules that can be the basis for separating molecules from one another in a mixture. As nonlimiting examples, agarose gels are known to be useful for separating polynucleotides and polyacrylamide gradient gels containing sodium dodecyl sulfate (SDS) are known to be useful for separating polypeptides, e.g., proteins. The matrix may be of uniform concentration throughout, such as a 1% agarose, which may be used to separate polynucleotides. Alternatively, the matrix may be a gradient, such as a 4-15% SDS-polyacrylamide gel for the separation of proteins. Other possible types of gels are known and may be used for the claimed method. One of skill in the art will be able to select a matrix appropriate for any desired application.

It is not a requirement of the claimed method that the matrix actually separates the molecules. For example, the claimed method may be performed by running an already homogeneous sample of molecules through a matrix and then transferring the molecules to the shrinkable film.

The Laminate

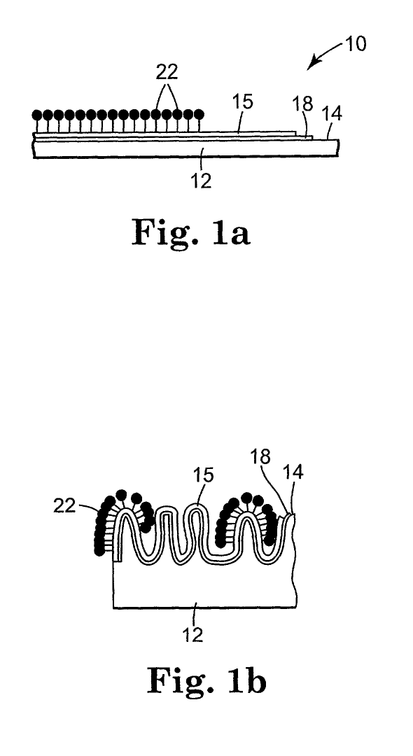

With reference to FIGS. 1a and 1b, the laminate 10 of the claimed method includes a substrate 12 with at least one major surface 14 having a surface area. The major surface 14 may be generally smooth or may include undulations. The substrate 12 may be any number of shapes. The shape of the substrate 12 is not limiting, so long as the substrate 12 provides a base for applying a surface coating 15 thereon, as described more fully below.

The substrate 12 is a shrinkable, polymeric material. Accordingly, the substrate 12 has a projected surface area and a topographical surface area. Prior to shrinking, the projected surface area and the topographical surface area are substantially equivalent. When shrunk, however, the substrate 12 may become undulated. In this case, the topographical surface area will be greater than the projected surface area.

In one embodiment, the surface coating 15 is at least partially adhered to the substrate 12 and has a generally smooth appearance. The surface coating 15 has a projected surface area and a topographical surface area. Accordingly, prior to shrinking the substrate 12, the projected surface area and the topographical surface area of the surface coating 15 are substantially equivalent.

As described more fully below, upon shrinking of the substrate 12, the topographical surface area of the surface coating 15 becomes greater than the projected surface area of the surface coating 15. The laminate 10 of the claimed method includes a surface coating 15 that is capable of exhibiting a topographical surface area that greatly exceeds the projected surface area. The topographical surface area of the surface coating 15 may be at least about five times greater than the projected surface area. In one embodiment, the topographical surface area is at least fifteen times greater than the projected surface area.

Upon shrinking of the substrate 12, the surface coating 15 may become undulated as depicted in FIG. 1b. While the undulations are irregular with respect to any discernable pattern, it is contemplated that a regular pattern of undulations may be obtained. The adhesion of the surface coating 15 to the substrate 12 should be sufficient to prevent its total delamination from the substrate 12. When the laminate 10 has an undulated surface, a degree of delamination may actually occur and still provide a useful laminate for use in the claimed method. However, the degree of delamination should not be so great as to interfere with assays being conducted on the laminate 10 or result in effective loss of the surface coating 15 from the substrate 12.

The laminate 10 of the claimed method is capable of exhibiting high topographical surface areas. The high topographical surface area offers opportunities for increasing the signal strength of certain assays. When shrunk, the undulated surface permits more molecules to be concentrated in a given projected surface area compared to transferring molecules to a relatively flat, unshrinkable surface. Also, in the case where transferred molecules are affixed prior to shrinking the substrate 12, the spatial relationship of the affixed molecules to one another on the surface is fixed. Upon shrinking of the substrate 12, the surface of the surface coating 15 becomes undulated, in effect increasing the density of affixed molecules with respect to the projected surface area but substantially maintaining their relative separation due to the topographical surface area of the surface coating 15. This spacing allows presentation of a high density of molecules at or near the surface of the surface coating 15 while minimizing potential steric crowding. This, in turn, facilitates rapid interaction kinetics with prospective assay reagents.

Substrates

The substrate 12 of the laminate 10 in the claimed method is a polymeric material. The material of the substrate 12 is selected with regard to the application for the resulting laminate. For example, if fluorescence will be used to detect the transferred molecules, the material used for the substrate 12 may be selected to exhibit low background fluorescence. Also, the substrate 12 material can be selected so that it is compatible with the reagents and conditions of the assays, such as temperature, solvents, and pH.

Many polymeric materials may be suitable for use in the laminate 10 of the claimed method. For certain embodiments having a high topographical surface area, one skilled in the art can select materials capable of being oriented, i.e., films that shrink at least in one direction within the film plane when energy such as heat is applied to the film for a specified period of time. Elastomeric materials are also suitable for use in the laminate 10 of the claimed method. Elastomeric materials include materials that are stretched in at least one direction prior to coating, constrained in the stretched state during coating, and then allowed to recover, thereby reducing the projected surface area of the substrate surface from the stretched state. Thus, herein, a relaxable substrate includes an oriented film and a recoverable substrate includes an elastomeric material.

With respect to oriented films, relaxation need not be equal in any two orthogonal directions within the film plane. In one embodiment, relaxation of the substrate 12, and therefore the laminate 10, is substantially uniform. In this embodiment, the oriented film relaxes in substantially the same amount in each direction, regardless of position on the film plane. If the oriented film employed does not exhibit substantially uniform relaxation characteristics, a registration indicator may be employed to register relative positions on the finished laminate.

The substrate 12 provides a surface upon which additional layers or other films or coatings (e.g., polymeric coatings, mask layers, etc.) may be disposed. Upon relaxation or recovery of the substrate 12, the substrate 12 provides support and integrity to the surface coating 15, or other films or coatings (e.g., polymeric coatings, mask layers, etc.) disposed thereon.

Oriented films suitable for use as a substrate 12 in the laminate 10 of the claimed method include, but are not limited to, biaxially oriented low-density polyethylenes, biaxially oriented linear low-density polyethylenes, and biaxially oriented ultra low-density polyethylenes. Biaxially oriented films exhibit shrinkage in two orthogonal in-plane directions (hereafter referred to as the “x” and “y” directions). Other oriented films that may be suitable for use in the claimed method include uniaxially, biaxially, or multiaxially oriented films made by any process known to the art, including, but not limited to: melt-orientation; the blown film, bubble, double-bubble, and tubular processes; length orientation; the process of tentering; extension over a mandrel; thermoforming; and blow molding. Polymers which may be employed in such films include, but are not limited to: polyethylenes, including high density polyethylene, low density polyethylene, linear low density polyethylene, ultra low density polyethylene, and copolymers of ethylene (including ethylene propylene copolymers and ethylene vinyl acetate copolymers); polyolefins, including isotactic polypropylene, syndiotactic polypropylene, and polymethylpentene; polyacetals; polyamides, including polyamide 6 and polyamide 66; polyesters, including polyethylene terephthalate, polybutylene terephthalate, and polyethylene naphthalate; halogenated polymers, including polyvinyl chloride, polyvinylidene chloride, polychlorotrifluoroethylene, polyvinyl fluoride, and polyvinylidene fluoride; styrene polymers, including general purpose polystyrene and syndiotactic polystyrene; cellulose esters, including cellulose acetate and cellulose propionate; polyketones, including polyetheretherketone and copolymers and terpolymers of carbon monoxide with ethylene and/or propylene; polycarbonates, including the polycarbonate of bisphenol A; phenyl-ring polymers, including polyphenylene sulfide; polysulfones; polyurethanes; polymers of acrylic and methacrylic acids and their esters; ionomers; and copolymers, blends, or layered structures of any of the above-named polymers. Oriented films of any of these polymers may be optionally cross-linked.

Examples of elastomeric materials that may be suitable for use as the substrate 12 in the laminate 10 of the claimed method include natural rubber, polyisoprenes, polychloroprene, polyisobutylenes, polybutenes, nitriles, polyurethanes, silicones, random copolymers and terpolymers (such as ethylene-propylene copolymers and ethylene-propylene-diene monomer terpolymers), and block copolymers.

Surface Coating

A surface coating 15 is at least partially adhered to the substrate 12 to form the laminate 10 of the present invention. A wide variety of surface coatings 15 may be suitable for use in the present invention. The surface coating 15 may include a hydrogel. As used herein, a hydrogel means a water-containing gel; that is, a polymer that is hydrophilic and will absorb water, yet is insoluble in water. The hydrogel provides a porous surface coating 15 capable of absorbing, for example, three to five times its dry weight in water. This provides a hydrophilic environment suitable for performing a wide variety of biological, chemical and biochemical assays on the transferred molecules.

In certain embodiments, the surface coating 15 may include linking agents 22 capable of immobilizing or affixing transferred molecules. If desired, more than one type of linking agent 22 may be used. When present, linking agents 22 can be an integral component of the coating 15, or can be affixed in a subsequent step to the surface coating 15, which is disposed on a substrate 12. Any number of processes known in the art may be used to introduce the linking agents 22 to be affixed to the surface coating 15. It is understood that the mode of affixation may vary in accordance with the linking agents 22 employed.

The type of linking agent 22 that may be used in the present invention may vary according to the application and the molecule to be detected or quantified. Linking agents 22 suitable for covalent immobilization of transferred molecules include azlactone moieties such as those provided by copolymers taught in International Publication No. WO 99/53319, published Oct. 21, 1999. Other useful linking agents 22 are also taught in the same publication. Azlactone moieties are useful because these moieties are suitable for reaction with many different molecules, including polypeptides, e.g., proteins. Azlactone moieties are generally hydrolytically stable and therefore have a relatively long shelf life when used in the applications of the invention. These moieties also generally exhibit high reactivity with transferred molecules or with other coatings comprising different linking agents. For example, additional coatings may be applied to provide other means of affixing the transferred molecules, such as ionic bonding.

The particular performance characteristics of the laminate 10 with respect to the assay(s) to be performed may be adjusted by varying the thickness of the surface coating 15. For example, fluorescence from a Western blot of transferred albumin and rabbit IgG was greater in laminates having a surface coating thickness of 10,000 Å compared to a laminate having a surface coating of 500 Å. One skilled in the art will be able to select a surface coating of an appropriate thickness to optimize the conditions for a desired assay.

Methods of Relaxation/Recovery and Functionalization

Relaxation and recovery of the films making up the substrate 12 can be accomplished using the methods described in International Publication No. WO 99/53319, published Oct. 21, 1999. Oriented films exhibit an area shrinkage reduction that is dependent in part on the degree of elongation of the film during orientation thereof. The area shrinkage reduction is a measure of the area shrinkage of the film from its oriented, pre-shrunken dimensions to its dimensions after energy has been applied to shrink the film. For example, a 10 cm×10 cm (100 cm² area) film that shrinks fifty percent (50%) in the “x” direction and fifty percent (50%) in the “y” direction after the application of sufficient heat will be reduced to 5 cm×5 cm (25 cm² area), thereby exhibiting an area shrinkage reduction of seventy-five percent (75%). An area shrinkage reduction of about twenty-five percent (25%) is suitable for the laminate 10 of the claimed method, but an area shrinkage reduction of more than about seventy-five percent (75%) may be achieved in certain embodiments, thereby producing a laminate with very high-densities of transferred molecules.

When miniaturization is desired, the substrate 12, and therefore the laminate 10, may be shrunk, i.e., a substrate 12 comprising an oriented film may be relaxed or a substrate 12 comprising a stretched elastomeric film may be recovered. The relative positions of the spots or bands occupied by the transferred molecules prior to shrinking the laminate should be maintained after the laminate is shrunk. However, the density of the transferred molecules may be increased dramatically.

With respect to oriented films, the reduction may be effected by the application of heat, although other modes of relaxing oriented films can be used. The mode of size alteration, such as the application of heat, can be selected so that it does not substantially impair the activity of the transferred molecules. For example, fairly high heat may be employed to shrink a substrate 12 having oligonucleotides affixed thereto (approximately 150 degrees Celsius) without destroying the ability to have subsequent DNA hybridization occur with the oligonucleotides.

With respect to elastomeric materials, the reduction of the projected surface area may be achieved by releasing the force that is holding the material in the stretched condition. The substrate 12 may be subsequently treated to hold the substrate 12 in the shrunken format. Alternatively, a backing or other physical means may be applied to the substrate 12 to hold it in the size-altered format.

The relative positions of the transferred molecules are maintained when the laminate 10 of the claimed method is shrunk. However, the density of the transferred molecules may be increased dramatically. Accordingly, the laminate 10 suitable for use in the claimed method may increase the density of the transferred molecules from the initial affixation of the molecules to the shrunken state by a substantial factor. Increases in the density of transferred molecules of 4-fold, 10-fold, and greater than 20-fold are possible according to the claimed method.

Increasing the density of the transferred molecules is advantageous where an intensified detection signal is desired, such as, for example, when fluorescent, absorbent, or chemiluminescent labels are used as detection signals. Moreover, the increase in density of the transferred molecules means that a smaller amount of the sample is required to elicit a signal substantially functionally equivalent, for example, to performing the same assay in a multi-well plate. Additionally, less assay media may be required to perform an assay on the reduced surface area occupied by molecules concentrated on the shrunken laminate 10 according to the claimed method compared to performing the same assay, for example, in a multi-well plate or on a non-shrinkable immobilizing membrane.

Additional Optional Features

In certain embodiments, the laminate 10 of the claimed method may include polymeric coatings, typically overlying the surface coating 15, if desired. Such polymeric coatings can provide a variety of linking agents on the surface coating 15. Alternatively, they can be applied to a surface coating 15 that already includes linking agents. Examples of polymeric coatings include those that are suitable for affixing reactants and are compatible with the assays and attendant conditions that are to be conducted on the particular film, such as those described in International Publication No. WO 99/55319, published Oct. 21, 1999.

In certain other embodiments, the device 10 may include an optional layer 18. The optional layer 18 may include a mask layer to reduce or prevent transmission of excitation energy through the mask layer to the underlying substrate 12, as disclosed in U.S. patent application Ser. No. 09/410,863, filed on Oct. 1, 1999. For other applications, a mask layer is used to reduce or prevent the transmission of electromagnetic energy from beneath the analyte, e.g., the substrate, that is similar to the electromagnetic signal emitted by the desired analyte in response to the excitation energy. In either case, with a mask layer in place, the electromagnetic signals emitted from the surface of the film can generally be attributed to excitation of the molecule captured on the film rather than the underlying substrate 12 or other portions of the film. As shown in FIG. 1a, certain embodiments will have the optional mask layer 18 underlying the surface coating 15.

With reference to FIG. 1a, the optional layer 18 may alternatively include an electromagnetic energy sensitive material, which may be the same or different than the material of the mask layer, if present. The optional layer 18 including electromagnetic energy sensitive material that is provided on the substrate 12 can take a variety of forms as described in U.S. patent application Ser. No. 09/459,418, filed on Dec. 9, 1999. Examples of some suitable materials include, but are not limited to, those described in U.S. Pat. Nos. 5,278,377 (Tsai); 5,446,270 (Chamberlain et al.); 5,529,708 (Palmgren et al.); and 5,925,455 (Bruzzone et al.). Although the optional layer 18 is depicted as being in direct contact with the substrate 12, one or more intervening layers may be located between the optional layer 18 and substrate 12 provided that the electromagnetic energy sensitive material, if present in the optional layer 18, is in thermal communication with the heat-relaxable material in the substrate 12 such that thermal energy in optional layer 18 is conducted to the substrate 12.

Transfer of Molecules to the Laminate

Molecules can be transferred from the matrix to the laminate 10 by any suitable process. For example, the molecules may be transferred from the matrix to the laminate 10 by passive blotting. The matrix may be, for example, a gel made from agarose or polyacrylamide through which a sample of molecules has been run and separated. The matrix is placed in contact with the laminate and the matrix and laminate 10 are assembled into a typical blotting configuration well known in the art, such as between layers of filter paper. Alternatively, the matrix and laminate are assembled in a commercially available blotting apparatus according to the apparatus manufacturer's instructions. During the blotting process, the molecules are transferred from the matrix to the laminate 10 in register with their positions in the matrix. Thus, the laminate 10 contains a replica of the pattern of molecules that was generated as the molecules were run through the matrix.

Alternatively, the molecules may be transferred from the matrix to the laminate 10 by electroblotting, i.e., blotting driven by an electric current. The matrix and the laminate 10 are assembled in an electroblotting apparatus and the apparatus is run according to the apparatus manufacturer's instructions. Once applied, the electric current drives the migration of the molecules from the matrix and onto the laminate 10. As in passive blotting, molecules are transferred to the laminate 10 in register with their relative positions in the matrix. Thus, an electroblotted laminate 10 will also contain a replica of the pattern of molecules that was generated as the molecules were run through the matrix.

Whichever transfer process is used, molecules from a single gel may be transferred to more than one laminate 10 according to the claimed method. Therefore, one can obtain a series of laminates, each with an identical replica blot of the pattern of molecules present in the matrix. The ability to obtain multiple identical blots from one matrix, according to the present invention, provide substantial advantages over the art for subsequent functional analysis of the transferred molecules. For example, one may produce a series of identical blots of a set of separated proteins from a sample comprising a mixture of proteins. One blot might be probed with one or more specific monoclonal antibodies, another one developed for carbohydrate functionality, another for a specific enzyme activity such as phosphatase or phosphorylase, or any of a number of other assays. After evaluating these various assays one would still have the matrix as a source for recovery of the untransferred proteins for further processing.

This is an exceptionally useful application of the claimed method as it allows one to perform several different analyses in parallel on replica blots of a single protein gel. Because each blot is in register with every other blot, individual proteins may be identified by their relative positions on each blot and those relative positions will be the same as the relative positions occupied by the proteins in the original gel. Thus, results from the series of parallel assays may provide data that can be used, for example, to identify or characterize individual proteins in the blots. Once identified or characterized, the position of any remaining protein in the original gel is known.

Additionally, because the proteins can be concentrated by shrinking the laminate 10 after the proteins are transferred, less of the protein may need to be transferred in order to perform the desired assays, thereby preserving more of each of the matrix-bound proteins for further processing, if desired. Also, less assay reagent may be needed to perform a particular assay on the transferred proteins after they have been concentrated as a result of shrinking the laminate 10, resulting in reduced costs. As an example, proteins from a 2-D protein gel may be transferred to a laminate 10, then shrunk to produce a replica that has, for example, a projected surface area 1/20^{th} that of the of the original gel. The shrunken laminate 10 thus may require a smaller volume of reagents to perform a particular assay compared to performing the same assay on proteins transferred to a non-shrinkable immobilizing membrane.

While characterized above with reference to separation, transfer, identification and analysis of proteins, one skilled in the art will recognize that similar advantages exist with respect to separation, transfer, identification and analysis of polynucleotides, polysaccharides or any other class of biological or non-biological molecules using the claimed method. Accordingly, the claimed method may be employed to transfer, identify and analyze polynucleotides or polysaccharides that have been run through a matrix in a manner similar to that described above for the transfer, identification and analysis of polypeptides, e.g., proteins.

### EXAMPLES

The following examples have been selected merely to further illustrate features, advantages, and other details of the invention. It is to be expressly understood, however, that while the examples serve this purpose, the particular ingredients and amounts used as well as other conditions and details are not to be construed in a matter that would unduly limit the scope of this invention.

### Example 1

Preparation of Fluorescein-Labeled Albumin

A 5 mg/mL solution of bovine serum albumin (BSA, available from Sigma Chemical Co., St. Louis, Mo.) in 0.1 M phosphate-buffered saline, pH 7.5 (PBS) was reacted with fluorescein isothiocyanate (Sigma-Aldrich Chemical Co., St. Louis, Mo.) (final concentration 1 mg/mL) at ambient temperature for 1 hour (total volume 1.0 ml), then dialyzed extensively against the phosphate-buffered saline. Analysis of the preparation indicated a protein concentration of 1.1 mg/mL with a fluorescein to albumin molar ratio of 0.65. The conjugate was stored at 4° C. protected from light with aluminum foil.

Fluorescein-labeled rabbit IgG (FITC-IgG) was purchased from Sigma Chemical Co.

Electrophoresis

Immediately prior to electrophoresis a sample of the fluorescein-protein conjugate was reduced and denatured with Laemmli reagent (Bio-Rad Corp., Hercules, Calif.) using standard techniques and electrophoresed through a 4-15% acrylamide gradient precast gel (Bio-Rad) at a constant voltage of 100 volts for 45 minutes. The gel was rinsed in sodium carbonate buffer (0.5 M, pH 9.0) for 10 minutes. Fluorescence was detected using a hand-held UV source.

Preparation of DMA-VDM Co-Polymer and Coating onto Shrink Film

Preparation of dimethylacrylamide/vinylazlactone (DMA-VDM) copolymer coating solutions was accomplished according to Example 10 of WO 99/53319. Solutions were diluted to 1.5% solids with isopropanol and formulated with enough ethylenediamine added immediately prior to coating to provide 10% crosslinking by weight. Coating was accomplished by use of wire-wound coating rods (Meyer bars). After coating, solvent was removed from the coating by placing the coated film in an oven heated to 50° C. for 30 minutes to provide a coating thickness of approximately 2000 Å. Other coating methods are described in WO 99/53319.

Western Blot

The rinsed electrophoresis gel was subjected to Western blotting on both nitrocellulose paper and polyethylene shrink film coated with azlactone copolymer. All materials except the coated shrink film were soaked in transfer buffer (48 mM Tris, 39 mM glycine, 20% methanol, pH 9.2) for 15-20 minutes. The coated shrink film was loaded with the azlactone surface facing the gel.

For the passive blotting experiments the materials were assembled in a Bio-Rad wet blotting apparatus according to the package insert and kept in contact overnight at ambient temperature without application of electricity.

In electroblotting the assembled sandwich was electrophoresed for 1 hour at 11 volts (256 mA) in a Bio-Rad blotting chamber maintained at 0-2° C. Additionally, semi-dry blotting was performed using a Bio-Rad semi-dry electrophoretic transfer cell (Model SD Cell) according to the package insert instructions.

Blotted films were dried and presence of the labeled protein was determined using a hand-held UV light source. Both albumin and IgG demonstrated distinct lines of fluorescence under UV illumination. Passive blotting and electroblotting each successfully transferred fluorescently-labeled protein to the shrink film. However, electroblotting transferred the fluorescently-labeled proteins to shrink film at a much higher rate than was obtained by passive blotting, indicating that electroblotting was considerably more efficient for transferring fluorescently-labeled proteins to coated shrink film.

### Example 2

Another standard gel, prepared and run as in Example 1, was electroblotted according to the procedure of that example four more times, in succession, from the single original polyacrylamide gel. The second and third blots produced strong fluorescent bands in the coated shrink film similar in intensity to that of the first blot. The fourth blot was reduced in intensity and the fifth blot yielded a lighter, but readily detectable, fluorescent intensity in the coated shrink film. The original gel still retained a strong glow of fluorescence after the fifth blot. These observation demonstrate that a gel can be blotted to several different films to produce identical replicas of the original gel as well as of one another.

### Example 3

Preparation of Different Thickness DMA-VDM Polymer Coatings

Shrink film was coated with DMA-VDM copolymer described above. To effect different thicknesses of the copolymer we used a combination of different concentration copolymer solutions and different Meyer bars. Coatings were produced with thicknesses of 500 Å, 1000 Å, 2500 Å, and 10,000 Å (1 μm).

Fluorescein-labeled albumin and FITC- IgG were run on the precast acrylamide gels as described in Example 1. The gels were blotted electrophoretically onto the coated shrink films and were allowed to dry.

All of the blots had detectable fluorescence, although the intensity of the fluorescence tended to generally increase with the thickness of the surface coating from a level that was readily detectable for the 500 Å coating to saturating brilliance for the two thickest coatings.

### Example 4

An FITC-labeled oligonucleotide (5′-FITC-AGGATTCCGGGTTAT available from Sigma Genosys, The Woodlands, Tex.) was dissolved in deionized water at concentrations of 56 μM, 111 μM, 222 μM, and 445 μM. An agarose electrophoresis gel (0.75%) was run with samples of this dilution series in adjacent lanes. Imaging of the gel by scanning on a 575 FLUORIMAGER (Molecular Dynamics, Sunnyvale, Calif.) showed fluorescent intensities expected for the different concentrations. A sample of polyethylene shrink film coated with a 1 μm thick layer of the azlactone copolymer described above was used as the blotting laminate. Blotting was accomplished as follows:

Transfer Procedure:

(a) Coated laminate was cut to a size that was slightly larger than the gel. A notch was cut on the laminate to align it with the gel and to provide orientation.

(b) Two pieces of filter paper were soaked in transfer buffer (18X SSC, 1M ammonium acetate) and placed on the top of a stack of absorbent filter paper. Any bubbles were removed.

(c) The laminate was placed on top of the filter paper.

(d) The gel was placed face down on top of the laminate and all bubbles were removed.

(e) A sheet of plastic wrap was placed on top of the gel. The plastic wrap was cut with a razor blade immediately next to the gel and the portion of the saran wrap immediately on top of the gel was removed. (This helps in preventing short-circuiting of the transfer buffer).

(f) Filter paper presoaked in transfer buffer was placed on top of the saran wrap. Any bubbles were removed.

(g) Sponges were soaked in transfer buffer and placed on top of the filter paper. The transfer was allowed to continue overnight.

(h) After completion of transfer, the laminate was removed and scanned on the FLUORIMAGER. Highly fluorescent images were seen in increasing intensities corresponding to the dilution series, indicating transfer of the oligonucleotide from the gel to the laminate.

The complete disclosures of the patents, patent documents and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method of transferring molecules positioned within a matrix to a laminate comprising:
contacting the matrix with a laminate comprising i) a substrate comprising a shrinkable polymeric film, and ii) a hydrogel disposed on the substrate, to transfer one or more molecules from the matrix to the laminate.

2. The method of claim 1 wherein the hydrogel comprises linking agents.

3. The method of claim 2 wherein the linking agents comprise azlactone copolymers.

4. The method of claim 1 wherein the laminate further comprises a mask layer.

5. The method of claim 4 wherein the mask layer is in direct contact with the substrate and underlies the hydrogel.

6. The method of claim 1 wherein the one or more molecules are transferred from the matrix to the laminate by electroblotting.

7. The method of claim 1 wherein the matrix contains polynucleotides, polypeptides, polysaccharides, or combinations thereof.

8. The method of claim 1 wherein the matrix comprises an agarose gel or a polyacrylamide gel.

9. The method of claim 1 further comprising detecting the one or more molecules transferred from the matrix to the laminate.

10. The method of claim 1 wherein the shrinkable polymeric film is flexible.

11. The method of claim 1 wherein the shrinkable polymeric film is heat-shrinkable.

12. A composition comprising:
a laminate comprising i) a substrate comprising a polymeric film and ii) a hydrogel disposed on the substrate, the laminate having a projected surface area and a topographical surface area wherein the topographical surface area is greater than the projected surface area; and
one or more molecules affixed to the laminate;
wherein the one or more molecules are configured so that they may be assayed or detected.

13. The composition of claim 12 wherein the laminate further comprises a mask layer.

14. The composition of claim 13 wherein the mask layer is in direct contact with the substrate and underlies the hydrogel.

15. The composition of claim 12 wherein the molecules are selected from polypeptides, polynucleotides, polysaccharides, and any combination thereof.

16. A method of preparing molecules positioned within a matrix for analysis comprising:
contacting the matrix with a shrinkable laminate having a projected surface area and a topographical surface area so that one or more molecules are transferred from the matrix to the laminate; and
shrinking the laminate so that the topographical surface area is greater than the projected surface area.

17. The method of claim 16 wherein the laminate comprises:
a substrate comprising a polymeric film, and
a hydrogel disposed on the substrate.

18. The method of claim 16 wherein the laminate further comprises a mask layer.

19. The method of claim 16 wherein the mask layer is in direct contact with the substrate and underlies the hydrogel.

20. The method of claim 16 wherein the laminate comprises linking agents.

21. The method of claim 20 wherein the linking agents comprise azlactone copolymers.

22. The method of claim 16 wherein the one or more molecules are transferred from the matrix to the laminate by electroblotting.

23. The method of claim 16 wherein the matrix contains polynucleotides, polypeptides, polysaccharides, or combinations thereof.

24. The method of claim 16 wherein the matrix comprises an agarose gel or a polyacrylamide gel.

25. The method of claim 16 further comprising detecting the one or more molecules transferred from the matrix to the laminate.
